(19) United States
(12) Patent Application Publication
(10) Pub. No.: US 2004/0138445 A1
(43) Pub. Date: Jul. 15, 2004

(54) Process for obtaining bio-functional fractions from biomass

(76) Inventor: Doug Van Thorre, Minneapolis, MN (US)

Correspondence Address: Schwegman, Lundberg, Woessner & Kluth, P.A., P.O. Box 2938, Minneapolis, MN, 55402, US

(21) Appl. No.: 10/670,178

(22) Filed: Sep. 24, 2003

Related U.S. Application Data
(63) Continuation-in-part of application No. 10/340,877, filed on Jan. 10, 2003.
Application No. 10/340,877 is a continuation of application No. PCT/US01/41322, filed on Jul. 10, 2001.

(51) Int. Cl. C07H 1/00; C07C 229/02

(52) U.S. Cl. 536/124; 562/402

(57) ABSTRACT

The present invention includes a method for extracting bio-functional and bio-responsive fractions from biomass. The method includes providing biomass; treating the biomass with saturated steam at a time and temperature effective to extract bio-functional fractions; rapidly depressurizing the biomass and steam; mixing a depressurized bio-functional fraction with reagent that breaks down the fraction into oligomers and monomers; and separating the monomers from the each other and the oligomers using ion exchange.

### RELATED APPLICATIONS

This application is a Continuation-In-Part of U.S. application Ser. No. 10/340,877 which is a Continuation under 35 U.S.C. 111(a) from International Application No. PCT/US01/41322, filed Jul. 10, 2001, and published in English at WO 02/04084 on Jul. 17, 2002, which claims priority from U.S. application Ser. No. 09/613,411, filed Jul. 10, 2000, which application and publications are incorporated by reference.

### BACKGROUND OF THE INVENTION

The present invention relates to a method for extracting bio-functional fractions from biomass that includes subjecting the biomass to high-frequency, rotor-stator shearing. One embodiment includes purifying the fractions using ion-exchange with no acid addition.

Extraction and purification of biologically active materials from biomass has been a complicated and inefficient endeavor. Extraction has traditionally employed harsh solvents; created intermediate reaction products and physical conditions very different from conditions forming the extracted chemicals in the first place. Because of these harsh conditions, there has been some question as to whether complex molecules such as native cellulose have ever really been extracted in a way that preserves the native cellulose structure.

This concern extends to the separation and purification of optically pure bio-functional materials. Drug and fine chemical feedstocks have been produced to exacting physical and chemical purity standards or chirality. However, little regard has been given to optical purity. Achieving optical purity requires identifying feed stock components that have stereoisomers and selecting D- or L-forms of chemicals that have stereoisomers. The D- and L-forms are known as stereoisomer pairs, i.e. right and left handed pairs. Stereoisomers are molecules that are identical in atomic constitution, and that have, in some instances essentially identical physical and chemical properties. The stereoisomeric pairs differ in three dimensional arrangement of atoms, optical rotation, and chemical properties.

One type of stereoisomer pair is an enantiomer. An enantiomer is a stereoisomer pair with at least one asymmetric center. Individual stereoisomers of an enantiomer are mirror images of each other. Drugs tend to have enantiomers that have activity which is biologically distinguishable. In some instances, individual enantiomers of drugs have distinguishable biological activity. Naturally occurring, optically impure, or racemic mixtures of stereoisomers have been used as feedstocks in the pharmaceutical and fine chemical industries. In many instances, the quality of the final product has been insensitive to the optical purity of the feedstock. However, in some cases, the chemical and optical purity of the final product has depended, in part, upon the optical purity of the feed stock.

One stereoisomeric drug having one enantiomer which shows a different biological activity in humans than the other enantiomer is d,l-propranolol. l-propranolol acts as a beta-blocker. d-propranolol lacks such activity.

In some instances, one of the enantiomers is toxic while the other enantiomer is benign. For instance, when a d-isomer was removed from d,l-carnitine in a drug composition, doctors could no longer observe symptoms of myasthenia gravis. Symptoms had been observed, however, in patients taking the racemic mixture of d,l-carnitine.

One other example is thalidomide. It is well known that ingestion of R,S-thalidomide in the 1950's by pregnant women led to the birth of children with phocomelia and other embryopathies. It was subsequently found that the R enantiomer of thalidomide is teratogenic and toxic in an animal model while the S enantiomer of thalidomide is neither teratogenic nor toxic in that model. Unfortunately, no benefit is found in humans of using the S enantiomer thalidomide over the R enantiomer because humans morph the pure S form to a racemic mixture of R,S-thalidomide. It is still unknown which enantiomer of thalidomide is toxic in humans. Therefore, thalidomide use is prohibited in most cases in the United States.

Because enantiomers have radically different biological activity, the FDA has developed a set of rules governing the development of stereoisomeric drugs. These rules can be found at the FDA web site. Specifically, the FDA requires that the enantiomeric composition of a drug should be known. That is, the stereochemical identity, strength, quality, and purity should be known in the final product. The FDA has further stated that “appropriate manufacturing and control procedures should be used to assure stereoisomeric composition of a product, with respect to identity, strength, quality and purity.” Thus, pharmaceutical feedstocks, and fine chemical feedstocks used to formulate products which come under the FDA regulatory power, must be produced with utmost concern for the chirality of the molecules.

One group of chemicals that is rich in stereoisomers is the group comprising carbohydrates. Conventional carbohydrate chemistry for extracting sugars from sugar cane pulp, bagasse, or sugar beet biomass requires using large amounts of caustic and hydrochloric acid to hydrolyze the cellulose and hemicellulose polymer backbone. In the extraction, the mixed carbohydrate biomass is initially placed into a caustic solution where it forms ellipsoidal aggregates. The typical formulation calls for approximately 100 pounds of caustic for each pound of hemicellulose/cellulose carbohydrate mixture. This extraction step is accompanied by disposal problems. Since the ellipsoidal aggregates are only weakly permeable to aqueous solutions, the hydrolysis process must be performed at high temperatures and for an extended period of time.

What occurs is thermal degradation of the exterior of the ellipsoidal aggregate before the hydrolysis reaction has traversed the radius of the aggregate. The degradation results in a diminished yield and a need to separate the degraded carbohydrate from the hydrolyzed hemicellulose/cellulose mixture. Conventional extraction requires a significant destruction of raw material due to thermal decomposition of the carbohydrate and environmental damage resulting from disposal of caustic and acidic process chemicals.

### SUMMARY OF THE INVENTION

The present invention includes a method for extracting bio-functional and bio-responsive fractions from biomass. The method includes providing or obtaining biomass; treating the biomass in a high-frequency, rotor-stator device to make sheared biomass; treating the sheared biomass with saturated steam at a time and temperature effective to extract bio-functional fractions; rapidly depressurizing the biomass and steam; mixing a depressurized bio-functional fraction with reagent that breaks down the fraction into oligomers and monomers; and separating the monomers from the each other and the oligomers.

Another embodiment of the present invention includes a process for extraction of monomers from biomass. The process includes obtaining or providing biomass; subjecting the biomass to steam at a time and temperature effective to extract the bio-functional materials comprising polymers; rapidly depressurizing the biomass to extract the bio-functional materials; mixing the bio-functional materials, in one or more static mixers, with one or more materials to hydrolyze the polymers to form hydrolysates; converting the hydrolysates to form a mixture of monomers, having no added acid; and separating the monomers from the mixture using ion exchange.

Another embodiment of the present invention includes a process for extracting a stereoisomer from biomass. The process includes providing or obtaining biomass; subjecting the biomass to substantially instantaneous pressurization and de-pressurization in a manner effective to separate lignin, hemicellulose and cellulose in the biomass; hydrolyzing the hemicellulose to form hemicellulose hydrolysates in a mixture free from added acid; and separating one or more stereoisomers from the hemicellulose hydrolysates using ion exchange.

One other embodiment of the present invention includes a system for obtaining monosaccharides, oligosaccharides and polysaccharides from biomass. The system includes a mechanism for substantially instantaneously pressurizing and de-pressurizing biomass to separate the biomass into hemicellulose, cellulose, and lignin; a heater for heating the hemicellulose to liquefy the hemicellulose; a static reactor/mixer for mixing a sodium hydroxide with hemicellulose and for making hemicellulose hydrolysates without an addition of acid; and ion exchange mechanisms comprising ion exchange resin for selectively separating a hemicellulose hydrolysate based upon the component's stereoisomeric identity.

### BRIEF DESCRIPTION OF THE FIGURES

FIG. 1 is a schematic view of one embodiment of the process of the present invention.

FIG. 2 is a schematic view of one process embodiment of the present invention.

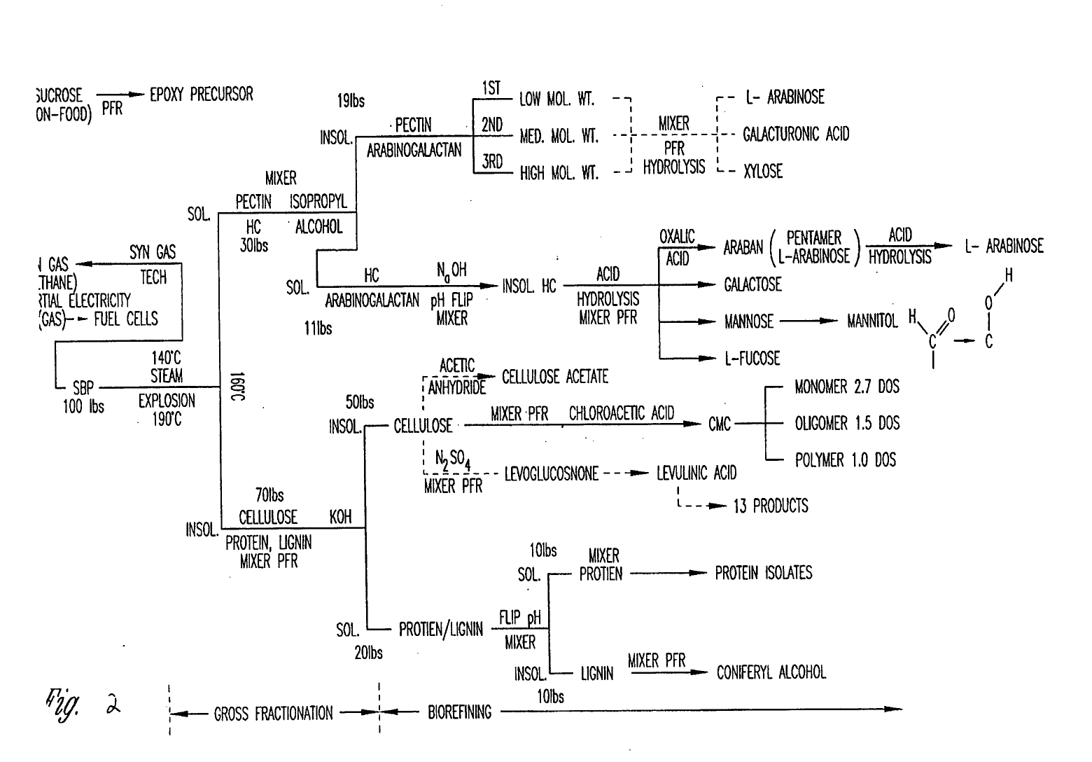

FIG. 3 is a schematic axial view of a high-frequency, rotor-stator shearing device used in the method of the present invention.

FIG. 4 is a schematic view of shear between rotor and stator of the rotor-stator shearing device illustrated in FIG. 3.

### DETAILED DESCRIPTION OF THE INVENTION

One embodiment of the present invention includes a method for extracting biofunctional fractions such as monomers, oligomers, and polymers from biomass in a manner effective for maintaining a bio-functionality and bio-response that is substantially the same as the materials had prior to extraction. The method includes subjecting a biomass substrate to high-frequency, rotor-stator shearing treatment in a supraton to form a supraton-treated slurry and then subjecting the supraton-treated slurry to saturated steam pressurization and depressurization.

The method of the present invention uses a high-frequency, rotor-stator shearing device in the treatment of biomass. This type of device produces high-shear, microcavitation forces which defibrillate the biomass fed into it. Two commercially available high-frequency, rotor-stator dispersion devices are the Supraton™ devices manufactured by Krupp Industrietechnik GmbH and marketed by Dorr-Oliver Deutschland GmbH of Connecticut, and the Dispax™ devices manufactured and marketed by Ika-Works, Inc. of Cincinnati, Ohio. These devices are mentioned to provide examples of suitable devices but are not meant to limit acceptable high-frequency, rotor-stator shearing devices usable in the processes of the present invention.

To prepare the biomass for shearing, the biomass is first reduced to a manageable size by grinding. Grinding to a desired particle size is accomplished in one or more stages. In a general aspect of the process, the milled biomass is ground by conventional hammermilling to a particle size sufficiently small enough to pass through a number 4 mesh sieve.

In one embodiment, the ground product is mixed with water to obtain a slurry of a preselected solids content. One of the purposes of this part of the process is to swell and further defibrillate the biomass. In one embodiment, the ground biomass is fed into a hopper and conveyed to a mixer-grinder-pump and water added to form a slurry having a solids content ranging from about 10% to about 25% solids. In one embodiment, the mixer-grinder-pump is a medium shear, rotorstator device capable of mixing and pumping high solid content slurries. This device further reduces the particle size of the biomass, wets the particles thoroughly with water, and disperses the particles within the water. Examples of this type of device are the HED™ manufactured and marketed by Ika Works, Inc. of Cincinnati, Ohio and the Gorator™ manufactured by Krupp Industrietechnik GmbH and marketed by Dorr-Oliver Deutschland GmbH of Connecticut.

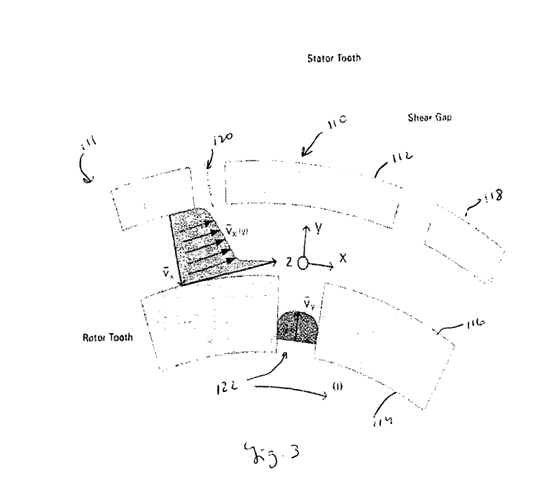

Referring to FIG. 3, a slurry is fed into the high-frequency, rotor-stator device 111 and is forced into a chamber 110. Inside the chamber is a series of coaxial meshing rings. The rings are configured with teeth, slots or bore holes. The rings configured with teeth are generally known as tooth and chamber tools and those configured with bore holes are generally known as nozzle tools. Generally, tooth and chamber tools are attached to both the rotor and the stator when tooth and chamber tools are used. When nozzle tools are used, generally, a tooth and chamber type tool is affixed to the rotor and a nozzle tool will be affixed to the stator.

The rings are concentric, radiating out from the center. The rings 112 on the stator are fixed and the rings 114 on the rotor are rotated by a shaft coupled to a motor.

The structure identified as 116 is representative of a tooth on a tooth and chamber tool attached to the rotor. The structure identified as 118 is representative of both a tooth on a tooth and chamber tool attached to the stator and the body of a nozzle tool spaced between bore holes. Accordingly, the space identified as 122 represents the gap between the teeth on a tooth and chamber tool attached to the rotor. And, the space identified as 120 represents both the gap between teeth on the stator tooth and chamber tool attached to the stator and the gap formed by a bore hole in a nozzle tool attached to the stator. The rings 114 on the rotor and the rings 112 on the stator are closely spaced at close tolerances. The space between the rotor and stator is typically about 1 mm.

Regarding a tooth and chamber tool, adjacent pairs of teeth are separated by gaps 120 and 122. The tooth and gap size determine the coarseness of the machine, i.e., a coarse tool has fewer teeth with larger gaps between adjacent teeth when compared with a medium or fine tool. Both the Supraton™ and Dispax™ allow the use of coarse, medium, and fine toothed rings in the same device, or the devices can have all coarse, all medium, or all fine toothed rings in the chamber so that the machines may be used in series, if desired. The use of multiple devices in series is one alternative to the use of a single device for processing biomass.

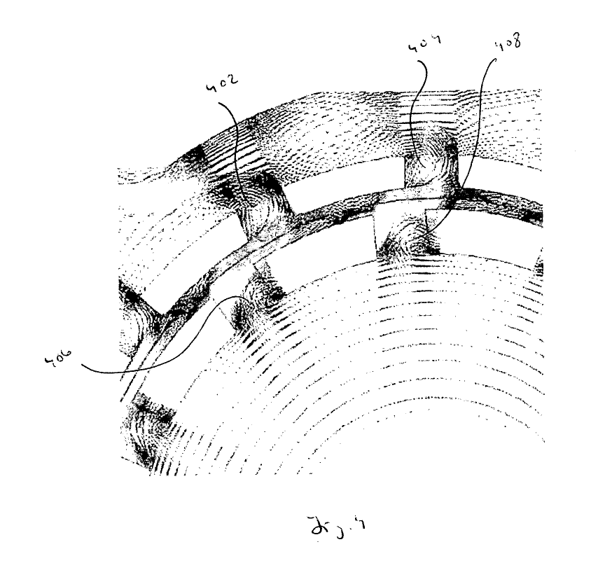

As the biomass slurry is pumped under pressure into the chamber 10 by the mixer-grinder-pump, the slurry encounters each concentric layer of the tools in place in the chamber as the slurry is forced laterally. This lateral force is created by the pressure on the slurry as it is pumped into the chamber by the mixer-grinder-pump and by the centrifugal force created by the spinning rotor. The slurry passes through the gaps between the teeth as the rotor spins past the gaps in the stator. Flow is most pronounced when the gaps 122 between the rotor teeth align with the gaps 120 in the stator. The result is a pulsing flow with a rapid succession of compressive and decompressive forces. The biomass material in the slurry is subjected to these repeated forces, as the centrifugal force accelerates it through the gaps toward the outer edge of the chamber. As the slurry moves towards the outer edge of chamber 110 the centrifugal forces increases, thus intensifying the forces generated in gaps 120 and 122. The repeated compressive and decompressive forces create microcavities in the slurry with extremely intensive energy zones. These zones are illustrated at 402, 404, 406 and 408 in FIG. 4. The biomass fibers are ripped apart by these forces. Additionally, the resulting fibers exhibit extensive internal decrystallization due to the forces generated in the microcavities.

As the biomass particles pass outward through the various gaps, the particles come in contact with the teeth and the body of the nozzle tool. Accordingly, some grinding of the particles may occur due to such contact. The grinding effects are relatively small, however, when compared with the combined effects of shear forces and microcavitation. Nonetheless, as solids loadings increase the instance of grinding may also increase.

Grinding typically cuts, slices, and dices fibrous material perpendicular to the fiber bundle, producing a more spherical type of particle. Shear forces in combination with microcavitation, on the other hand, tend to shatter the material, that is, they rip the fibers apart from the inside-out explosively forming irregularly shaped particles. Examination of these particles show them to have been “cut” both perpendicular to the fiber axis and longitudinally along the fiber axis. The effect on the fibers is to shatter their structure, possibly disrupting the cellulose bonding to hemicellulose without the compressive effects of grinding. Solids loadings not exceeding 30% are employed to minimize grinding of the biomass and thus the compressive effects of the grinding.

While the precise mechanisms occurring within the chamber of the high-frequency, rotor-stator device are not totally understood several factors are thought to aid in the explaining the effects on the treated biomass. The swelling effect of liquids, particularly water, is thought to aid in creation of longitudinal shearing effects in the treated biomass. The repeated compressive and decompressive events in and between the gaps are thought to create internal pressures tending to explode the biomass particles and thus the fibrous structure thereof. It is also hypothesized that a harmonic resonance effect may be created during operation of the rotor-stator device in the sonic range. Thus, a harmonic frequency of a particular fiber length when reached during processing would cause the effected fibers to resonate and tend to aid in the destruction of the fibrous structure of the biomass.

As previously stated, high-frequency, rotor-stator dispersion devices may have differently configured rings or “tools” within the chamber. These tools, for example, may vary in the gap size between the teeth on the rings or in bore hole size in the case of a nozzle tool. With a larger gap size, the resulting material is more coarse than with a smaller gap size. As stated earlier, these tools can be varied within one device to contain coarse, medium, and fine rings in the chamber of the device. Likewise, a device may contain rings of the same rating so that the devices can be staged. This capability is important for use in a continuous process.

Once the biomass is treated by a high-frequency, rotor-stator dispersion device, the treated biomass is subjected to pressurization and depressuriation. The process of pressurization and depressurization with saturated steam fractionates biofunctional materials to form hydrolysates-from hydrolysate fractions having the highest water content to hydrolysate fractions having the lowest water content. In one embodiment, the order of hydrolysate fractionation is extractibles such as terpenes, lignin, pectin, hemicellulose and native cellulose. What is meant by biofunctional and bio-reactive is that the tertiary and quaternary structures of these materials are not destroyed.

Once extracted, the fractions are cooled to ambient temperature. For some embodiments, the fractions are kept hot for further processing. With this method, there is a minimal loss in biofunctionality and bioresponse, as compared to traditional wet chemistry methods of separation. What is remarkable and unexpected is that this biofunctionality and bioresponse is achieved without complicated chemical treatment. Separation without loss of functionality and response is achieved by a one step steam pressurization/depressurization. What is also remarkable is that the extraction occurs with virtually any biomass feedstock. Pretreatment of biomass is minimal and is typically limited to size reduction.

The fractions are then subjected to ion exchange for the final purification. It has surprisingly been found that ion exchange is performed with no acid addition. Dissolved cations in fractions prepared in this manner are reduced to less than 100 ppm. Conventional wet chemistry fractions have a dissolved mineral cation concentration of several thousand ppm and higher. In one embodiment, an oligomer fraction was hydrolyzed to form a fraction with one or more monomers. The dissolved mineral cations in the monomer fraction were 4 weight percent. The analysis on the cations in the monomer fraction was as follows: magnesium, 2.5 ppm; calcium, 3.66 ppm; potassium, 0.40 ppm; and sodium, 1.81 ppm. The monomers were separated by ion exchange using ion exchange beads that were styrene crosslinked with divinyl benzene.

It is believed that with this treatment, substantially native physical and chemical properties and structure are preserved for molecules such as native cellulose. It is also believed that with this treatment, a mass balance can be performed over a plant for virtually all of the bio-functional materials within the plant. Products extracted are in a concentration and having a reactivity within a range of what is predictable from a mass balance.

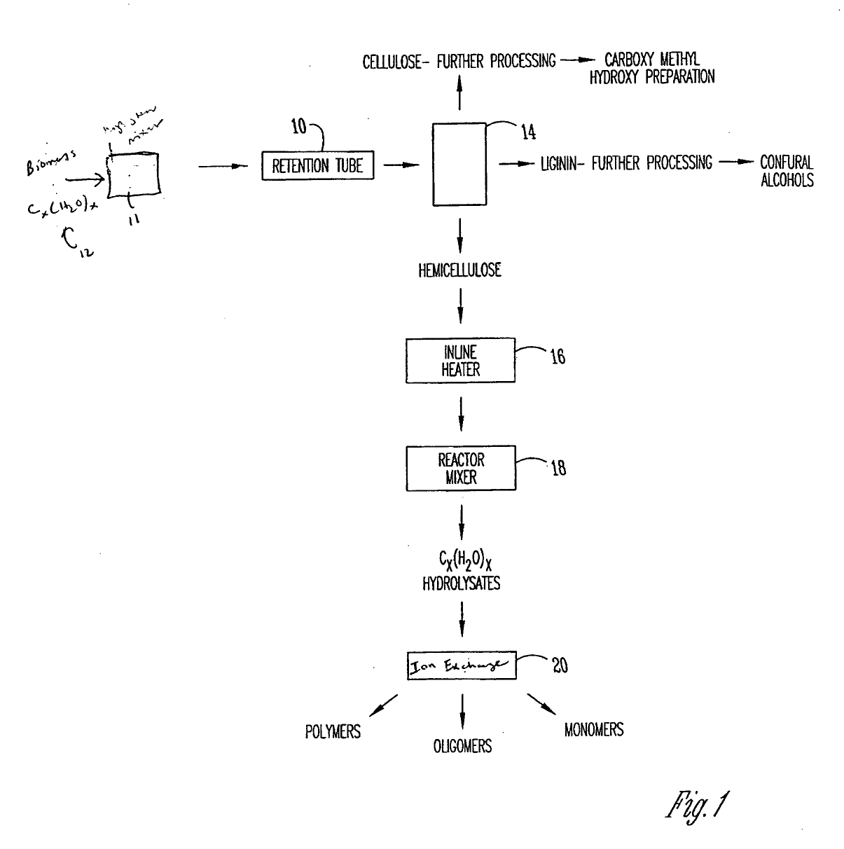

The present invention also includes a process for extracting, separating, and purifying individual stereoisomers and other specialty chemicals from biomass. The process, illustrated schematically at 10 in FIG. 1, comprises providing a source of biomass 12, subjecting the biomass to saturated steam pressurization/depressurization 14 that increases surface area of the biomass and that permits separation of lignin, cellulose, and hemicellulose components from the biomass, heating hemicellulose 16 separated from the biomass in order to hydrolyze the hemicellulose and obtain hydrolyzed monomers, oligomers and polymers, and separating polymers, oligomers, and monomers from hydrolyzed hemicellulose 18. While hydrolysates are described, it is understood that the process of the present invention is usable to extract, separate and purify substituents and derivatives of cellulose, hemicellulose and lignin. For instance, cellulose derivatives such as carboxy methyl cellulose and hydroxypropyl cellulose can be obtained using the process of the present invention. Coniferyl alcohols are also obtainable. Stereoisomers of the monomers are further extracted using chromatographic methods 20.

The present invention achieves high yields of stereoisomers, such as L-arabinose, using physical processes in addition to hydrolytic reactions, rather than exclusively conventional, water based, chemical extraction techniques. It has surprisingly been found that employing heat and pressure in treating biomass, such as sugar beet pulp or wood pulp, increases production rates and percent yield of stereoisomers as compared to conventional, water based, chemical extraction processes.

As used herein, “simple sugars” refer to monosaccharides and oligosaccharides which are not decomposed into smaller sugars upon hydrolysis. Monosaccharides include pyranoses and furanoses. Monosaccharides are also classified according to the number of carbons in the molecule; for example, d,l-arabinose is a heptose.

As used herein, “complex sugars” refer to polysaccharides which are carbohydrates of high molecular weight capable of being hydrolyzed into a large number of monosaccharide units. Typical polysaccharides are cellulose, lignin, hemicellulose, starch and pentosan.

An oligosaccharide is a simple polysaccharide with a known number of constituent monosaccharide units, such as 1 to 10 monomers.

The term “biomass” as used herein refers to plant materials including, but not limited to sugar beet pulp, bagasse, straw, corn stalks, corn cobs, grain husks, grass, and wood. Biomass in the form of plant materials includes cellulose and hemicellulose, both of which are polysaccharide, and lignin. Cellulose molecules are linear and unbranched glucose polymers with a high degree of polymerization between 10 and 10⁶. Cellulose has a strong propensity to form both intermolecular and intramolecular hydrogen bonds. Cellulose is stable against degradation under most physical and chemical conditions. Hemicellulose comprises heteropolysaccharides which are formed by a variety of different monomers. Most commonly the monomers are glucose, galactose, mannose, xylose and arabinose. Hemicellulose molecules have a degree of polymerization of about 10⁶. Biomass also includes entire plants, including stalk, roots, fruit, and so forth. The entire plants include but are not limited to corn plants, sugar beet plants, soy plants, wheat plants, cranberry plants, potato plants, sorghum plants, alfalfa plants, flax plants, and so forth

The term “feedstock” as used herein refers to any material supplied to a device, machine, or processing plant.

The biomass used in the process of the present invention may be obtained from a variety of processes that extract products from wood, sugar beets, corn, soy, wheat and any other plant matter. The biomass is subjected to a particle size of reduction to a size of chips or finer, such as a size of sawdust, using conventional particle reduction equipment. The smaller the size, the easier it is to mechanically handle the biomass. Smaller sized particles have a greater surface area and are more amenable to chemical reaction. Also, desired processing temperatures are reached more rapidly when using smaller particles.

In one embodiment, the biomass is fed into the high-frequency, rotor-stator device, illustrated schematically at 11 in FIG. 1 and is forced into a chamber 110, shown in FIG. 3. Inside the chamber is a series of coaxial meshing rings. The biomass treatment in the high-frequency, rotor-stator device is described above.

In one embodiment, the biomass is fed to a hopper following treatment in the rotor-stator device. The biomass may optionally be sprayed with water either before transfer to the hopper or while in the hopper. The biomass exits from the bottom of the hopper into a conveying feeder which contains a conveying mechanism such as a feed screw driven by a variable feed drive. The feed screw or other conveying mechanism feeds the material into a compacting feed tube and then into a pressurized retention tube, where the biomass particles are formed into a solid plug of material. The solid plug is compressed by surface pressures of up to 2000 psi.

The biomass is mechanically compacted prior to its introduction into the digester. The biomass is desirably in a moistened condition. The mechanical compaction removes air from the material prior to its introduction to steam pressurization. Air is undesirable because oxygen in the air tends to oxidatively degrade the biomass. Air also exerts a partial pressure and retards temperature and pressure equalization within the reactor.

Steam pressurization, within the pressurized reaction vessel, is typically operated with automatic pressure and temperature control systems. The partial pressure of any air pockets decreases steam pressure and temperature in the reactor below a preselected value. Compaction, followed by processing conditions discussed below, causes a degree of fibrillation of the biomass. Fibrillation of biomass assists in the heat transfer within and around the material.

Next, the biomass particles are disintegrated by steam pressure treatment and defibrination. In particular, the particles are treated with saturated steam at a temperature of from about 160 to 230 degrees Centigrade for a period of time from 2 minutes to 4 hours. The biomass is disintegrated by this steam treatment. In general, the lower the temperature used, the longer the duration of treatment should be. Thus, for some extractions, it is desirable to treat a biomass at 160 degrees Centigrade for about 4 hours. For other extractions, it is desirable to treat a biomass for 2 minutes at 230 degrees Centigrade.

This steam treatment separates fractions within biomass by most to least water content. The fractions are separated as extractables such as terpenes, fatty acids and so forth, lignin, pectin, hemicellulose and native cellulose. This steam treatment yields fractions at yields that are predictable by a mass balance of the biomass. In other words, the steam treatment and extraction of the present invention permits a user to ascertain bioactive/biofunctional materials present in living biomass and to extract the bioactive/biofunctional materials in quantities that approach or are substantially the same as the materials are present in the native biomass.

Biomass disintegrated this way is then, subsequently, for some embodiments, lixiviated with an aqueous solution of alkali. The concentration of NaOH is typically no greater than about 4% by weight.

The biomass mixture contains between 1 and 20 grams of water per gram of dry biomass and preferably about 16 grams of water per gram of dry biomass. In one embodiment the biomass mixture contains between 2 and about 50 grams of calcium hydroxide per 100 grams of dry biomass and preferably contains 30 grams of calcium hydroxide per 100 grams of dry biomass. In another embodiment the biomass mixture contains between 2 and 50 grams of alkali, hydroxide of sodium or hydroxide of potassium, per 100 grams of dry biomass.

The steam pressure treatment is performed in either a continuous stream or a batch type steam pressure reactor. In one embodiment, the reactor is manufactured by Stake Technology Ltd. Of Ottawa, Canada. One particular device is described in U.S. Pat. No. 4,136,207, which issued Jan. 23, 1979, and which is herein incorporated by reference. The steam pressure treatment is performed in the reactor vessel. The reactor vessel is maintained at a pressure that is between about 200 and 450 psig. The temperature in the reactor is maintained between about 390° F. and 460° F. The biomass is fed intermittently for some embodiments and continuously for other embodiments. By varying the biomass stream but maintaining the reactor vessel conditions, the method of the present invention introduces an efficiency to the process, by avoiding ramp up and ramp down conditions within the reactor vessel.

The biomass is introduced into the reaction vessel in a manner that forms a solid plug at the inlet of the vessel. In one embodiment, the solid plug is formed in a device, such as a retention tube. The biomass plug prevents a loss of pressurization in the vessel. The combination of the biomass plug and constant pressurization permits instantaneous steam penetration of the biomass within the reaction vessel, and thus permits better control of processing times.

The biomass is processed at the steam temperatures described for a period of at least about 15 seconds and for some embodiments, at least about 5 minutes. The maximum time is about one hour.

After cooking, the biomass is cooled and depressurized substantially instantaneously. The biomass is in a moisture saturated condition. The biomass is subjected to sudden and substantially instantaneous decompression and adiabatic expansion, e.g. by discharging a small quantity of cooked biomass into ambient conditions.

The process of instantaneous pressurization and de-pressurization separates the biomass into components of lignin, cellulose and hemicellulose. The hemicellulose product is separated from the cellulose product and lignin product by techniques known in the art. It is further contemplated that the cellulose product is separated from the lignin product by techniques in the art.

Once the hemicellulose is extracted from the biomass, the hemicellulose for some embodiments, is heated in a steam heater, such as a Komax steam heater and then is hydrolyzed in a static mixer, such as a Komax reactor/static mixer, manufactured by Komax Systems, Inc., of Long Beach, Calif. One reactor/static mixer embodiment is described in U.S. Pat. No. 6,027,241, which is herein incorporated by reference. The reactor/static mixer is, in one embodiment, constructed so that an additive, such as sodium hydroxide is added countercurrent to the main fluid stream. The heater and mixer comprise a heater-mixer system.

Within the reactor, at approximately 329° F. hemicellulose undergoes a phase transition, depending upon the moisture content, from a solid to a non-Newtonian fluid, somewhat like tooth paste. At temperatures higher than approximately 500° F., depending upon moisture content, the hemicellulose begins to pyrolize. Furthermore, the xylan component of the hemicellulose is degraded at temperatures above 428° F. Hence, to preserve the quality of the hemicellulose product stream, the hemicellulose exposure to temperatures above 356° F. should be as short as possible. The in-line reactor heater—static mixer system raises the temperature of the hemicellulose to between 329° F. and 347° F. The time to bring the temperature within this range is typically less than about 10 seconds to about 20 seconds.

Once heated, the hemicellulose is reacted with NaOH in the reactor/static mixer. The static mixer accepts the hemicellulose, a high viscosity stream and NaOH, the low viscosity stream. The NaOH is injected into the high viscosity stream, mixed by static mixing and a chemical reaction occurs between the alkali and the hemicellulose. In particular, the NaOH hydrolyzes the hemicellulose. The process of the present invention, unlike conventional sugar extraction processes, does not rely upon chemical reactions for extraction. Instead, the process of the present invention utilizes both sophisticated mechanical separation, occurring in the static mixer, coupled with NaOH addition for hydrolysis, for extraction and formation of hydrolysates.

In one embodiment, the hydrolysates include dissolved solids at a concentration of about four weight percent. The specific cation analysis included magnesium in a concentration of 2.5 ppm; calcium in a concentration of 3.66 ppm; potassium in a concentration of 0.40 ppm and sodium in a concentration of 1.81 ppm. The hydrolysate is prepared at 205 degrees Centigrade for three minutes with no acid.

The oligomers in the hydrolysates are converted to monomers without the addition of acid and are separated, in some embodiments, by ion exchange. In one embodiment, ion exchange columns are one inch in diameter and contain resin that is 3-4 feet deep. Ion exchange beds include a first cation resin bed and a second bed. Ion exchange beads are styrene crosslinked with divinyl benzene. Flow through the ion exchange bed is at 60 to 80 degrees Centigrade and is retained in the bed for a fifteen minute retention time. There is then a total retention time of thirty minutes through both of the beds. The conversion of the oligomers to monomers in the hydrolysate is then measured. If conversion not at a preselected value, the hydrolysate effluent is heated to 80 degrees Centigrade and samples are taken until conversion is completed. In one embodiment, the liquor provided to the ion exchange beds is a mixture of oligomers (3/5) and monomers (2/5).

If acidity is insufficient to complete the conversion, acid is added to the cation effluent to drive the reaction. The cation effluent pH is typically within a range of 2.5 to 3.5. Post hydrolysis is performed mildly with acid and once oligomers are converted to monomers, the reaction mixture is concentrated by vacuum evaporation to 50 percent dissolved solids.

With the process of the present invention, the hydrolysate mixture is further hydrolyzed to the basic monomeric unit from oligomers, and polymers in a single step and then separated on the basis of stereoisomer, i.e. optical or chirally pure monomer separation, in a second step. In another embodiment, the hydrolysate mixture is separated and a desired stereoisomer may be extracted in a single step.

In one particular embodiment of the process of the present invention, L-arabinose is extracted from biomass, the source of which is sugar beet pulp. The sugar beet pulp is transported from a sugar beet process stream to a chopper or grinder and then to a hopper. From the hopper, the chopped or ground beet pulp is transported to a retention tube by a conveyor such as a feed screw. Within the retention tube, the sugar beet pulp is formed into a solid plug.

The solid plug is transferred to the steam pressurized reactor where it is disintegrated by defibrination. The reaction temperature is 160 to 230 degrees Centigrade and the time period is about 2 to 10 minutes. Upon disintegration, the biomass is substantially instantaneously depressurized by removal from the reaction.

This process separates the cellulose, lignin and hemicellulose from each other. The hemicellulose is separated and is passed through the heater-reactor/static mixer system described above. Arabinose is one of the sugar hydrolysates produced.

In one embodiment of the process of the present invention, sugar products obtained by ion exchange are crystallized. In one embodiment, the crystallization is performed using a low intensity ultrasonic agitation. It is believed that this crystallization produces a product wherein crystals have few inclusions, are uniform in shape, in size, in density and in purity.

In one embodiment, the L-arabinose is separated from other monomers using the ion exchange methods and resin described herein. In another embodiment, the L-arabinose is separated from a mixture of hemicellulose hydrolysates.

For some embodiments, the high-frequency, rotor-stator treatment is used in a process that includes the ion exchange embodiment. For other embodiments, the process includes one of either the high frequency, rotor-stator treatment or the ion exchange embodiment.

The present invention may be embodied in other specific forms without departing from its spirit or essential characteristics. The described embodiments are to be considered in all respects only as illustrative and not restrictive. The scope of the invention is, therefore, limited only by the appended claims rather than by the foregoing description. All changes which come within the meaning and range of functional equivalency of the claims are to be embraced within their scope.

What is claimed is:

1. A method for extracting bio-functional and bio-responsive fractions from biomass, comprising:
providing or obtaining biomass;
treating the biomass in a high-frequency, rotor-stator device to make sheared biomass;
treating the sheared biomass with saturated steam at a time and temperature effective to extract bio-functional fractions;
rapidly depressurizing the biomass and steam;
mixing a depressurized bio-functional fraction with reagent that breaks down the fraction into oligomers and monomers; and
separating the monomers from the each other and the oligomers.

2. The method of claim 1 wherein the biomass is subjected to pressurization at a temperature of about 390 to 460 degrees Fahrenheit.

3. The method of claim 1 wherein the biomass is subjected to pressurization for a time ranging from 2 minutes to 4 hours.

4. The method of claim 1 wherein the separation material used in monomer separation includes styrene crosslinked with divinyl benzene.

5. A process for extraction of monomers from biomass, comprising:
obtaining biomass;
subjecting the biomass to saturated steam at a time and temperature effective to extract the bio-functional materials comprising polymers;
rapidly depressurizing the biomass to extract the bio-functional materials;
mixing the bio-functional materials, in one or more static mixers, with one or more materials to hydrolyze the polymers to form monomeric and oligomeric hydrolysates;
converting the hydrolysates to form a mixture of monomers, having no added acid; and
separating the monomers from the mixture using ion exchange.

6. The process of claim 5 wherein the ion exchange includes media comprising beads that include styrene crosslinked with divinylbenzene.

7. A process for extracting a stereoisomer from biomass, comprising:
providing biomass;
subjecting the biomass to substantially instantaneous pressurization and depressurization in a manner effective to separate lignin, hemicellulose and cellulose in the biomass;
hydrolyzing the hemicellulose to form hemicellulose hydrolysates in a mixture free from added acid; and
separating one or more stereoisomers from the hemicellulose hydrolysates using ion exchange.

8. The process of claim 7 and further comprising reducing size of the biomass prior to pressurization.

9. The process of claim 7 and further comprising compacting the biomass prior to pressurization.

10. The process of claim 7 wherein the biomass provided is one or more of wood, beets, corn, soy, wheat, and plant biomass.

11. The process of claim 7 wherein the stereoisomer separated is L-arabinose.

12. The process of claim 7 wherein the biomass is subjected to pressurization at a temperature of about 390 to 460 degrees Fahrenheit.

13. The process of claim 7 wherein the biomass is subjected to pressurization for not more than about 10 minutes.

14. The process of claim 7 wherein the biomass is reduced to a size sawdust.

15. The process of claim 7 and further comprising feeding the biomass for pressurization continuously.

16. The process of claim 7 and further comprising adding moisture to the biomass before pressurization.

17. The process of claim 1 wherein the hydrolysis occurs in a reactor/static mixer.

18. The process of claim 7 wherein sodium hydroxide is added to the static mixer in a flowpath that is counter-current to the flow of hemicellulose.

19. A system for obtaining monosaccharides, oligosaccharides and polysaccharides from biomass, comprising:
a mechanism for substantially instantaneously pressurizing and depressurizing biomass to separate the biomass into hemicellulose, cellulose, and lignin;
a heater for heating the hemicellulose to liquefy the hemicellulose;
a static reactor/mixer for mixing a sodium hydroxide with hemicellulose and for making hemicellulose hydrolysates without an addition of acid; and
ion exchange mechanisms comprising ion exchange resin for selectively separating a hemicellulose hydrolysate based upon the component's stereoisomeric identity.

20. The system of claim 1 the ion exchange resin comprises styrene crosslinked with divinyl benzene.

21. The system of claim 19 wherein the hemicellulose product passes through a glass transition state and into its liquefied state very rapidly.

22. The system of claim 15 wherein the hemicellulose hydrolysates comprised arabinose, l-arabinose, d-xylose, l-xylose, d-glucose, l-glucose, and any other racemic carbohydrates.
